(19) United States
(12) Patent Application Publication
(10) Pub. No.: US 2021/0161851 A1
(43) Pub. Date: Jun. 3, 2021

(54) COMBINATION THERAPY OF LYMPHOMA

(71) Applicant: Deutsches Krebsforschungszentrum, Heidelberg (DE)

(72) Inventors: Tabea Schlör, Hannover (DE); Jan Nicolay, Mannheim (DE); Peter Krammer, Heidelberg (DE); Karsten Gülow, Heidelberg (DE)

(21) Appl. No.: 17/107,099

(22) Filed: Nov. 30, 2020

Related U.S. Application Data
(60) Provisional application No. 62/941,907, filed on Nov. 29, 2019.

(51) Int. Cl. A61K 31/225; A61P 35/00; A61K 31/496; A61K 31/5377

(52) U.S. Cl. CPC A61K 31/225; A61K 31/5377; A61K 31/496; A61P 35/00

(57) ABSTRACT

The present invention relates to an NF-κB inhibitor or a pharmaceutically acceptable salt or prodrug thereof for use in treating lymphoma, wherein said use further comprises administration of at least one agent activating the intrinsic pathway of apoptosis; and to an agent activating the intrinsic pathway of apoptosis or a pharmaceutically acceptable salt or prodrug thereof for use in treating lymphoma, wherein said use further comprises administration of at least one NF-κB inhibitor. The present invention further relates to combined preparations, medicaments, kits, and methods related thereto.

The present invention relates to an NF-κB inhibitor or a pharmaceutically acceptable salt or prodrug thereof for use in treating lymphoma, wherein said use further comprises administration of at least one agent activating the intrinsic pathway of apoptosis; and to an agent activating the intrinsic pathway of apoptosis or a pharmaceutically acceptable salt or prodrug thereof for use in treating lymphoma, wherein said use further comprises administration of at least one NF-κB inhibitor. The present invention further relates to combined preparations, medicaments, kits, and methods related thereto.

Lymphomas are cancers of the lymphatic system, causing tumors in lymph nodes, the skin, the spleen, or in other parts of the lymphatic system. The main types of lymphoma are Hodgkin lymphoma and Non-Hodgkin lymphoma. Hodgkin lymphoma is characterized histopathologically by the presence of multinucleated Reed-Stemberg cells in tumor preparations. Non-Hodgkin lymphoma is the generic term used for all lymphomas which are not Hodgkin lymphoma, including diffuse large B-cell lymphoma, follicular lymphoma, marginal zone lymphoma, peripheral T-cell lymphoma, cutaneous T-cell lymphoma, small cell B-cell lymphoma, and mantle cell lymphoma.

The most commonly used—and in many instances the only approved or available—systemic treatment for cancer is chemotherapy. For patients suffering from lymphoma or metastases of solid tumors chemotherapy, thus, frequently is the only treatment option. Chemotherapeutic agents are cytotoxic for all rapidly dividing cells. As cancer cells usually divide more rapidly than other cells in the body, they are preferably killed by these agents. Common groups of chemotherapeutic agents are substances that inhibit cell division by interfering with the formation of the mitotic spindle or agents which damage the DNA, e.g. by alkylating nucleobases. Because all rapidly dividing cells are targeted by chemotherapeutic agents, their side effects are usually severe. Depending on the substance used, adverse effects include organ toxicity (e.g. heart or kidney), immunosuppression, neurotoxicity, and anemia. Some groups of chemotherapeutic agents, e.g. alkylating agents, even have the potential to cause cancer. Due to these side effects dosages have sometimes to be reduced or chemotherapy has to be discontinued completely. Furthermore, the side effects of chemotherapy often prohibit the treatment of patients in bad general condition. Adding to all these problems is the often limited efficacy of chemotherapy. In some cases chemotherapy fails from the very beginning. In other cases tumor cells become resistant during the course of treatment. To combat the emergence of resistant tumor cells and to limit the side effects of chemotherapy, combinations of different compounds with different modes of action are used.

An important mode of action of chemotherapeutic agents is the induction of apoptosis. Many chemotherapeutic agents, e.g. alkylating agents, crosslinking agents or antimetabolites induce DNA damage which finally leads to apoptosis of the affected cells. The often poor efficacy of chemotherapeutic agents in tumor cells can be explained by the disruption of normal apoptotic pathways in these cells. Cells in many tumors, for instance, lack a functional copy of p53. The product of this gene is responsible for controlling the cell cycle and initiating DNA-repair in the case of DNA damage. In cells with large scale DNA damage, p53 induces apoptosis. Without a functional p53 gene cells progress through the cell cycle and proliferate despite DNA-damage.

Apoptosis pathways involve diverse groups of molecules. One set of mediators implicated in apoptosis are so-called caspases, cysteine proteases that cleave their substrates specifically at aspartate residues. Caspases convey the apoptotic signal in a proteolytic cascade, with caspases cleaving and activating other caspases which subsequently degrade a number of target death proteins, such as poly(ADP-ribose)polymerase, eventually resulting in cell death. If one or more steps in this cascade are inhibited in tumor cells, these cells fail to undergo apoptosis and, thus, continue to grow. Caspase activation itself can be triggered by external stimuli affecting certain cell surface receptors, known to the person skilled in the art as so-called death receptors. Known death receptors mediating apoptosis after reception of an extrinsic signal include members of the tumor necrosis factor (TNF) receptor superfamily such as CD95 (APO-1/Fas) or TRAIL (TNF-related apoptosis inducing ligand) receptors 1 and 2. Stimulation of the death receptor CD95 leads to the formation of a cell membrane death inducing signaling complex (DISC, comprising CD95, FADD, pro-caspase 8 and c-FLIP) and among others, to the activation of caspase-8, which in turn activates other caspases and members of another group of apoptosis mediators.

In an alternative pathway of apoptosis induction known as intrinsic pathway of apoptosis induction, apoptosis is induced by an intracellular stress response via the mitochondria leading to the release of mitochondrial proteins. Extensive DNA damage is one of the factors that activate the intrinsic apoptotic pathway. Several Bcl-2 family members, commonly referred to as anti-apoptotic members of the Bcl-2 family, are thought to inhibit the release of the mitochondrial proteins and, thus, prevent cells from undergoing apoptosis.

Consequently, over-expression of the anti-apoptotic Bcl-2 family proteins Bcl-2, Bcl-xL, Bcl-w, and Mcl-1 (myeloid cell leukemia 1 protein) are frequently associated with tumor initiation, progression and resistance to conventional chemotherapies (Giam et al., 2009, Oncogene 27 Suppl 1:S128-36). These anti-apoptotic Bcl-2 proteins bind to the pro-apoptotic proteins Bak (Bcl-2 antagonist/killer) and Bax (Bcl-2-associated X protein) to prevent cell death. Only when Bak and Bax are released from their anti-apoptotic counterparts, they cause cell death by inducing the release of cytochrome c and activation of caspase-9 and -3. Thus, the anti-apoptotic proteins of the Bcl-2 family are validated drug targets for cancer treatment (Lessene et al., 2008, Nat Rev Drug Discov 7:989-1000). However, some tumor entities tend to overcome sensitivity to Bcl-2 family inhibitors by overproducing one or more anti-apoptotic Bcl-2 proteins, frequently Mcl-1, or by overproducing the X-linked inhibitor of apoptosis (XIAP), which prevents apoptosis at the effector phase by binding to and inhibiting activated caspase-3 and caspase-9, i.e. downstream of the anti-apoptotic Bcl-2 proteins.

ABT-737 and the orally bio-available ABT-263 (Navitoclax®) and ABT-199 (Venetoclax®) are small-molecule mimetics of the Bcl-2 homology domain 3, which inhibit Bcl-2, Bcl-xL and Bcl-w with high affinities. ABT-737, ABT-263, and ABT-199 have been shown to be effective as single agent in hematological malignancies and also in other types of cancers (Lessene et al., 2008, Nat Rev Drug Discov 7:989-1000). Currently, ABT-263 is being investigated in clinical trials in patients with lymphoid malignancies and small cell lung cancer. However, inhibition of Bcl-x_{L} by ABT-737/263 induces a concentration-dependent decrease in the number of circulating platelets (Tse et al., 2008, Cancer Res 68:3421-8). This side effect limits the ability to increase drug concentrations into a higher efficacious range.

The small molecule compound Dimethylfumarate (DMF) has been used in the treatment of psoriasis for decades and approved in Germany since 1995. Recently, DMF was identified as an NF-κB inhibitor inducing apoptosis in cutaneous T cell lymphoma (CTCL) cells (Nicolay et al. (2016), Blood 128(6):805) and a clinical trial for use of DMF in CTCL was initiated (ClinicalTrials.gov Identifier: NCT02546440).

Nonetheless, there is an urgent need to develop novel options to improve lymphoma treatment, in particular treatment of CTCL. In particular, there is a need to provide means and methods avoiding at least in part the drawbacks of the prior art as discussed above.

This problem is solved by the compounds for use, the preparations, medicaments, kits, and methods with the features of the independent claims. Preferred embodiments, which might be realized in an isolated fashion or in any arbitrary combination are listed in the dependent claims.

Accordingly, the present invention relates to an NF-κB inhibitor or a pharmaceutically acceptable salt or prodrug thereof for use in treating lymphoma, wherein said use further comprises administration of at least one agent activating the intrinsic pathway of apoptosis.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “NF-κB inhibitor” is understood by the skilled person to relate to any compound decreasing downstream signaling by NF-κB in a host cell, irrespective whether the compound inhibits activation of NF-κB, intracellular translocation of NF-κB, and/or interaction of NF-κB with upstream or downstream signaling components of the cell, including DNA. Methods for determining NF-κB inhibitor activity are known in the art and involve in particular measurement of the promoter activity of one or more NF-κB-target genes. Preferably, the NF-κB inhibitor is a fumarate ester, more preferably a bis-ester, still more preferably a bis-C1 to C5 alkyl ester of fumarate. Most preferably, the NF-κB inhibitor is dimethyl-fumarate (DMF, Dimethyl (E)-butenedioate, CAS No. 624-49-7).

The term “pharmaceutically acceptable salt” is understood by the skilled person to relate to any salt which is not detrimental to the active compound, to other compounds comprised in a preparation, and/or to the recipient thereof. Preferred acceptable salts are acetates, sulfates, chlorides, and the like.

The term “derivative”, as used herein, relates to a compound similar in structure to the compound it is derived from. Preferably, a derivative is a compound obtainable from a compound of interest by at most three, preferably at most two, more preferably by one derivatization step(s) known to the skilled person. More preferably, a derivative is a compound obtainable from a compound of interest by at most three, preferably at most two, more preferably by one derivatization step(s) selected from (i) alkylation, preferably N- and/or O-alkylation, preferably methylation, ethylation, propylation, or isopropylation; (ii) esterification, preferably of —COOH and/or —OPO₃H₂ groups, preferably acetylation, propionylation, iso-propionylation, or succinylation; (iii) amidation, preferably acetamidation; (iv) reduction, preferably of C═C, hydroxyl, and/or carbonyl groups; (v); oxidation, preferably of hydroxyl, C—H, and/or C—C groups. More preferably, a derivative is an N-methyl or N-ethyl derivative, a carboxylic acid acetate or succinylate, or an N-acetyl derivative.

The term “prodrug” is understood by the skilled person to relate to a compound not having or having only to a reduced extent the relevant activity as specified and being converted in the body of a subject to the actual active compound. Thus, preferably, a prodrug is a derivative of the active compound which is transformed, preferably cleaved, more preferably hydrolyzed, in the body of a subject to the active compound as specified above. More preferably, the prodrug is an ether, an ester, a glycosylate, a phosphate, a sulphate, or a macromolecule-conjugated, e.g. polyethyleneglycol (PEG) conjugated, derivative of the compound as indicated. More preferably, the prodrug is an ether, still more preferably an ester, of the active compound. Moreover, the prodrug may be a stereoisomer of the active compound which is isomerized in the body of a subject to the active compound.

The term “lymphoma” relates to a type of cancer developing from lymphocytes. Preferably, lymphocytes are T-cells, i.e., the lymphoma preferably is a T-cell lymphoma. More preferably, the lymphoma is a cutaneous T cell lymphoma (CTCL). CTCL is known to the skilled person as a class of non-Hodgkin lymphoma derived from T cells. Symptoms and forms of CTCL are known in the art and include non-specific dermatitis, enlarged lymph nodes, enlarged liver and spleen, mycosis fungoides or Sezary syndrome, and migration of mutated T-cells into the skin.

The term “agent activating the intrinsic pathway of apoptosis”, as used herein, relates to a chemical compound modulating the intrinsic pathway of apoptosis in a way that a cell contacted with said compound undergoes apoptosis, wherein said cell preferably is a cell insensitive to normal induction of apoptosis. The term “normal induction of apoptosis” is known to the skilled person and relates to any treatment or condition causing apoptosis to occur in a normal cell, preferably a non-tumor cell, most preferably in platelet cells, peripheral blood T cells, peripheral blood B cell, bone marrow stem cells, or in cardiac muscle cells. Preferably, the compound activating the intrinsic pathway of apoptosis is an inhibitor of the interaction of at least one member of the Bcl-2 family of proteins with its or their natural ligand or ligands. Preferably, said anti-apoptotic members of the Bcl-2 family of proteins are selected from the group consisting of Bcl-2, Bcl-x_{L}, and Bcl-w. More preferably, the compound activating the intrinsic pathway of apoptosis is a mimetic of the Bcl-2 homology domain 3 (BH3 domain). Such molecules and means of identifying them are known in the art and have been summarized, e.g. in Lessene et al., 2008, Nat Rev Drug Discovery 7: 989.

Preferably, the compound activating the intrinsic pathway of apoptosis is selected from the list consisting of a stapled peptide derived from a Bcl-2-interacting protein, in particular SAHBA, which is a stapled peptide derived from Bcl-2-interacting mediator of cell death (Walensky et al., 2004, Science 305: 1466); a Terphenyl derivative (Yin et al., 2005, J Am Chem Soc 127(29): 10191), in particular the derivative of the formula (I)

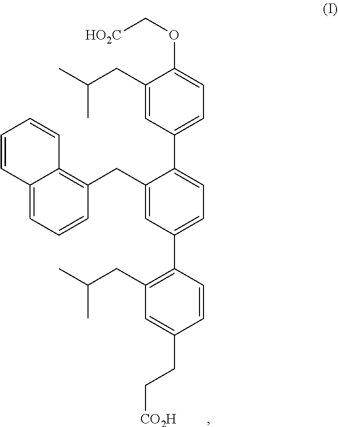 [SMILES: CC(C)Cc1cc(-c2ccc(-c3ccc(CCC(=O)O)cc3CC(C)C)cc2Cc2cccc3ccccc23)ccc1OCC(=O)O]

a Benzoylurea derivative (US 2008/153802), an Isooxazolidine (WO 2008/060569), and A-385358 (Wendt et al., 2006, J Med Chem 49:1165). More preferably, the compound activating the intrinsic pathway of apoptosis is selected from the list consisting of ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide), ABT-737 (4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide), and ABT-199 (4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)-Benzamide. Most preferably, the compound activating the intrinsic pathway of apoptosis is ABT-199.

The term “treating” refers to an amelioration of the diseases or disorders referred to herein or the symptoms accompanied therewith to a significant extent. Said treating as used herein also includes an entire restoration of the health with respect to the diseases or disorders referred to herein. It is to be understood that treating may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

As referred to herein, treatment comprises administration of at least the two pharmaceutically active compounds indicated to a subject in a concomitant treatment. Thus, preferably, at least the two pharmaceutically active compounds are administered such that said compounds are both present in the subject at an effective concentration for at least 25%, more preferably at least 50%, still more preferably at least 75% of the time in which the compound more slowly removed from the body is present at an effective concentration. Preferably, independent from the mode of administration, the NF-κB inhibitor and the agent activating the intrinsic pathway of apoptosis are present in the subject simultaneously at an effective concentration for at least 1 hour per treatment cycle, preferably at least 2 hours per treatment cycle, more preferably at least 3 hours per treatment cycle. Also preferably, independent from the mode of administration, the NF-κB inhibitor and the agent activating the intrinsic pathway of apoptosis are present in the subject simultaneously at an effective concentration for at least 25% of the time of a treatment cycle, preferably at least 50% of the time of a treatment cycle, more preferably at least 75% of the time of a treatment cycle, most preferably at least 85% of the time of a treatment cycle. Preferably, a treatment cycle is the repeating time unit of administration; preferably, administration is at least daily, more preferably is at least twice daily, still more preferably is at least three times daily, even more preferably is at least four times a day, most preferably is four times a day. Accordingly, a treatment cycle may be as long as several weeks; preferably the duration of a treatment cycle is of from 3 h to 24 h, more preferably of from 4 h to 12 h.

Preferably, administration is separate or combined administration. “Separate administration”, as used herein, relates to an administration wherein at least two of the pharmaceutically active compounds are administered via different routes and/or at different parts of the body of a subject. E.g. one compound may be administered by enteral administration (e.g. orally), whereas a second compound is administered by parenteral administration (e.g. intravenously). Preferably, separate administration comprises administration of at least two physically separated preparations, wherein each preparation contains at least one pharmaceutically active compound; said alternative is preferred e.g. in cases where the pharmaceutically active compounds of the combined preparation have to be administered via different routes, e.g. parenterally and orally, due to their chemical or physiological properties. Conversely, “combined administration” relates to an administration wherein the pharmaceutically active compounds of the present invention are administered via the same route, e.g. orally or intravenously. Preferably, administration is combined administration, more preferably combined oral administration.

Also preferably, administration is simultaneous or sequential administration. “Simultaneous administration”, as used herein, relates to an administration wherein the pharmaceutically active compounds are administered at the same time, i.e., preferably, administration of the pharmaceutically active compounds starts within a time interval of less than 15 minutes, more preferably, within a time interval of less than 5 minutes. Most preferably, administration of the pharmaceutically active compounds starts at the same time, e.g. by swallowing a tablet comprising the pharmaceutically active compounds, or by swallowing a tablet comprising one of the pharmaceutically active compounds and simultaneous injection of the second compound, or by applying an intravenous injection of a solution comprising one pharmaceutically active compound and injecting the second compound in different part of the body. Conversely, “sequential administration”, as used herein, relates to an administration causing plasma concentrations of the pharmaceutically active compounds in a subject enabling the synergistic effect of the present invention, but which, preferably, is not a simultaneous administration as specified herein above. Preferably, sequential administration is an administration wherein administration of the pharmaceutically active compounds, preferably all pharmaceutically active compounds, starts within a time interval of 1 or 2 days, more preferably within a time interval of 12 hours, still more preferably within a time interval of 4 hours, even more preferably within a time interval of one hour, most preferably within a time interval of 5 minutes. Preferably, administration is simultaneous administration, more preferably simultaneous oral administration. Preferably, administration is simultaneous combined administration, more preferably is simultaneous combined oral administration of a solid preparation, in particular a tablet comprising at least the two pharmaceutically active compounds referred to herein.

Preferably, the NF-κB inhibitor is DMF and the DMF is administered at a daily dose of at most 500 mg, preferably at most 450 mg, more preferably at most 400 mg. Also preferably, the NF-κB inhibitor is DMF and the DMF is administered at a daily dose of from 300 mg to 500 mg, preferably of from 350 mg to 475 mg, more preferably of from 375 to 450 mg for at least three, preferably at least four, more preferably at least six, weeks. Also preferably, the NF-κB inhibitor is DMF and the DMF is administered at a daily dose of at most 7 mg/kg body weight, preferably at most 6 mg/kg body weight, more preferably at most 5.5 mg/kg body weight. Also preferably, the NF-κB inhibitor is DMF and the DMF is administered at a daily dose of from 4 mg/kg body weight to 7 mg/kg body weight, preferably of from 4.5 mg/kg body weight to 6 mg/kg body weight, more preferably of from 5 mg/kg body weight to 5.5 mg/kg body weight for at least three, preferably at least four, more preferably at least six, weeks. Also preferably, the NF-κB inhibitor is DMF and the DMF is administered at a daily dose of from 100 mg to 300 mg, preferably of from 150 mg to 275 mg, more preferably of from 175 to 250 mg for at least three, preferably at least four, more preferably at least six, weeks. Also preferably, the NF-κB inhibitor is DMF and the DMF is administered at a daily dose of at most 3.5 mg/kg body weight, preferably at most 3 mg/kg body weight, more preferably at most 2.5 mg/kg body weight. Also preferably, the NF-dB inhibitor is DMF and the DMF is administered at a daily dose of from 1 mg/kg body weight to 4 mg/kg body weight, preferably of from 1.5 mg/kg body weight to 3.5 mg/kg body weight, more preferably of from 2 mg/kg body weight to 3 mg/kg body weight for at least three, preferably at least four, more preferably at least six, weeks.

Preferably, the agent activating the intrinsic pathway of apoptosis is ABT-199, ABT-263, or ABT-737, more preferably is ABT-199, and is administered at a daily dose of at most 500 mg, preferably at most 450 mg, more preferably at most 400 mg. Also preferably, the agent activating the intrinsic pathway of apoptosis is ABT-199, ABT-263, or ABT-737, more preferably is ABT-199, and is administered at a daily dose of from 300 mg to 500 mg, preferably of from 350 mg to 475 mg, more preferably of from 375 to 450 mg for at least three, preferably at least four, more preferably at least six, weeks. Also preferably, the agent activating the intrinsic pathway of apoptosis is ABT-199, ABT-263, or ABT-737, more preferably is ABT-199, and is administered at a daily dose of at most 7 mg/kg body weight, preferably at most 6 mg/kg body weight, more preferably at most 5.5 mg/kg body weight. Also preferably, the agent activating the intrinsic pathway of apoptosis is ABT-199, ABT-263, or ABT-737, more preferably is ABT-199, and is administered at a daily dose of from 4 mg/kg body weight to 7 mg/kg body weight, preferably of from 4.5 mg/kg body weight to 6 mg/kg body weight, more preferably of from 5 mg/kg body weight to 5.5 mg/kg body weight for at least three, preferably at least four, more preferably at least six, weeks. Also preferably, the agent activating the intrinsic pathway of apoptosis is ABT-199, ABT-263, or ABT-737, more preferably is ABT-199, and is administered at a daily dose of from 100 mg to 300 mg, preferably of from 150 mg to 275 mg, more preferably of from 175 to 250 mg for at least three, preferably at least four, more preferably at least six, weeks. Also preferably, the agent activating the intrinsic pathway of apoptosis is ABT-199, ABT-263, or ABT-737, more preferably is ABT-199, and is administered at a daily dose of at most 3.5 mg/kg body weight, preferably at most 3 mg/kg body weight, more preferably at most 2.5 mg/kg body weight. Also preferably, the agent activating the intrinsic pathway of apoptosis is ABT-199, ABT-263, or ABT-737, more preferably is ABT-199, and is administered at a daily dose of from 1 mg/kg body weight to 4 mg/kg body weight, preferably of from 1.5 mg/kg body weight to 3.5 mg/kg body weight, more preferably of from 2 mg/kg body weight to 3 mg/kg body weight for at least three, preferably at least four, more preferably at least six, weeks.

Preferably, the NF-κB inhibitor is DMF, the agent activating the intrinsic pathway of apoptosis is ABT-199, ABT-263, or ABT-737, and the molar ratio of agent activating the intrinsic pathway of apoptosis to DMF is at least 0.1, preferably at least 0.12, more preferably at least 0.14, most preferably at least 0.15, for at least three, preferably at least four, more preferably at least six, weeks. Also preferably, the NF-κB inhibitor is DMF, the agent activating the intrinsic pathway of apoptosis is ABT-199, ABT-263, or ABT-737, and the molar ratio of agent activating the intrinsic pathway of apoptosis to DMF is of from 0.1 to 0.3, preferably of from 0.12 to 0.2, more preferably is about 0.17, most preferably is 0.17, for at least three, preferably at least four, more preferably at least six, weeks. Also preferably, the NF-κB inhibitor is DMF, the agent activating the intrinsic pathway of apoptosis is ABT-199, ABT-263, or ABT-737, and the mass ratio of ABT-199 to DMF is at least 0.75 for at least three, preferably at least four, more preferably at least six, weeks. Also preferably, the NF-κB inhibitor is DMF, the agent activating the intrinsic pathway of apoptosis is ABT-199, ABT-263, or ABT-737, and the mass ratio of ABT-199 to DMF is of from 0.75 to 2, preferably of from 0.75 to 1.25, more preferably is about 1, most preferably is 1, for at least three, preferably at least four, more preferably at least six, weeks.

Advantageously, it was found in the work underlying the present invention that combination therapy of lymphoma with an NF-κB inhibitor and an agent activating the intrinsic pathway of apoptosis improves treatment outcome by a synergistic effect of the two compounds on cancer cells. Moreover, it was surprisingly found that as a consequence of the synergism between the two compounds, the dose, in particular the dose of the NF-κB inhibitor, may be reduced while maintaining the effect on cancer cells, which decreases the risk of adverse effect occurring during treatment.

The definitions made above apply mutatis mutandis to the following. Additional definitions and explanations made further below also apply for all embodiments described in this specification mutatis mutandis.

The present invention further relates to an agent activating the intrinsic pathway of apoptosis or a pharmaceutically acceptable salt or prodrug thereof for use in treating lymphoma, wherein said use further comprises administration of at least one NF-κB inhibitor.

The present invention also relates to a combined preparation for simultaneous, separate or sequential use comprising

a) at least one NF-κB inhibitor or a pharmaceutically acceptable salt or prodrug thereof; and
b) at least one agent activating the intrinsic pathway of apoptosis or a pharmaceutically acceptable salt or prodrug thereof.

The term “combined preparation”, as referred to in this application, relates to a preparation comprising the pharmaceutically active compounds of the present invention in one preparation. Preferably, the combined preparation is comprised in a container, i.e. preferably, said container comprises all pharmaceutically active compounds of the present invention. Preferably, said container comprises the pharmaceutically active compounds of the present invention as separate formulations, i.e. preferably, one formulation of the NF-κB inhibitor and one formulation of the agent activating the intrinsic pathway of apoptosis. As will be understood by the skilled person, the term “formulation” relates to a, preferably pharmaceutically acceptable, mixture of compounds, comprising or consisting of at least one pharmaceutically active compound of the present invention. Preferably, the combined preparation comprises an NF-κB inhibitor and an agent activating the intrinsic pathway of apoptosis in a single, preferably solid, pharmaceutical form, e.g. a tablet. Preferably, one or both of the pharmaceutically active compound(s) of the present invention is/are comprised in an immediate or fast release formulation. Preferably, the combined preparation is for separate or for combined administration as specified herein above. Also preferably, the combined preparation is for simultaneous or for sequential administration as specified herein above.

The present invention also relates to the combined preparation as specified herein for use in medicine, preferably for use in the treatment of lymphoma.

The present invention further relates to a medicament comprising

a) at least one NF-κB inhibitor or a pharmaceutically acceptable salt or prodrug thereof; and
b) at least one agent activating the intrinsic pathway of apoptosis or a pharmaceutically acceptable salt or prodrug thereof
and at least one pharmaceutically acceptable carrier.

The present invention also relates to medicament as specified herein for use in medicine, preferably for use in the treatment of lymphoma.

The term “medicament”, as used herein, relates to a combined preparation as specified herein above comprising the indicated pharmaceutically active compounds for combined and simultaneous administration as specified herein above and further comprises at least one pharmaceutically acceptable carrier. The pharmaceutically active compounds of the present invention can be formulated as pharmaceutically acceptable salts and/or as prodrug as specified elsewhere herein. Suitable routes of administration conventionally used for drug administration are oral, intravenous, subcutaneous, or parenteral administration as well as inhalation. However, depending on the nature and mode of action of a compound, the pharmaceutical compositions may be administered by other routes as well. The combined preparations or medicaments are, preferably, administered topically or, more preferably, systemically. Moreover, the compounds can be administered in combination with other drugs either in a common pharmaceutical composition or as separated pharmaceutical compositions as specified elsewhere herein, wherein said separated pharmaceutical compositions may be provided in form of a kit of parts. Preferably, the medicament is a single solid pharmaceutical form, e.g. a tablet. Preferably, the medicament is for oral administration, more preferably is a gastroprotected, solid, oral preparation, i.e., preferably, is coated to provide gastric acid resistance.

The pharmaceutically active compounds are, preferably, administered in conventional dosage forms prepared by combining the drugs with standard pharmaceutical carriers according to conventional procedures. These procedures may involve mixing, granulating and compressing or dissolving the ingredients as appropriate for the desired preparation. It will be appreciated that the form and character of the pharmaceutically acceptable carrier or diluent is dictated by the amount of active ingredient with which it is to be combined, the route of administration and other well-known variables. The carrier(s) must be acceptable in the sense of being compatible with the other ingredients of the formulation and being not deleterious to the recipient thereof. The pharmaceutical carrier employed may be, for example, a solid, a gel or a liquid. Exemplary of solid carriers are lactose, terra alba, sucrose, talc, gelatin, agar, pectin, acacia, magnesium stearate, stearic acid, degradable polymers like PLGA (DeYoung at al. (2011), Diabetes Technology & Therapeutics 13:1145; Ramazani et al., (2016), Int J Pharm. 499(1-2): 358-367), and the like. Exemplary liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. The diluent(s) is/are selected so as not to affect the biological activity of the compound or compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers, reactive oxygen scavengers, and the like.

Therapeutically effective doses of the pharmaceutically active compounds are described elsewhere herein. In case the medicament is a solid dosage form, such as a tablet, each dose preferably comprises at most a daily dose of the pharmaceutically active compounds, more preferably at most half a daily dose, even more preferably at most a third of a daily dose, most preferably at most a fourth of a daily dose. Thus, preferably, the medicament, preferably is a dosage form, preferably a tablet, for daily, preferably twice daily, more preferably three times daily, most preferably four times daily, administration.

The dosage regimen will be determined by the attending physician and other clinical factors; preferably in accordance within the specification described herein above. As is well known in the medical arts, dosages for any one patient depends upon many factors, including the patient's size, body surface area, age, the particular compound to be administered, sex, time and route of administration, general health, and other drugs being administered concurrently, as well as occurrence and severity of adverse effects. Progress can be monitored by periodic assessment.

The present invention also relates to a kit comprising (i) at least one NF-κB inhibitor or a pharmaceutically acceptable salt or prodrug thereof and at least one agent activating the intrinsic pathway of apoptosis or a pharmaceutically acceptable salt or prodrug thereof; (ii) a combined preparation as specified herein, and/or (iii) a medicament as specified herein, comprised in a housing.

The term “kit”, as used herein, refers to a collection of the aforementioned components. Preferably, said components are combined with additional components, preferably within an outer container, i.e. a housing. The outer container, also preferably, comprises instructions for carrying out a method of the present invention. Examples for components of the kit as well as methods for their use have been given elsewhere herein. The kit, preferably, contains the aforementioned components in a ready-to-use formulation. Preferably, the kit additionally comprises instructions, e.g., a user's manual for applying the components. Details are to be found elsewhere in this specification. Additionally, such user's manual may provide instructions about correctly using the components of the kit. A user's manual may be provided in paper or electronic form, e.g., stored on CD or CD ROM. The kit preferably comprises a means for administering at least one of its components. The skilled person knows that the selection of the means for administering depends on the properties of the compound to be administered and the way of administration. Where the compound is or is comprised in a liquid and the mode of administration is oral, said means, preferably, is a drinking aid, such as a spoon or a cup. In case the liquid shall be administered intravenously, the means for administering may be an i.v. equipment.

The present invention also relates to a method of inhibiting a lymphoma cell, comprising

a) contacting said lymphoma cell with (i) at least one NF-κB inhibitor or a pharmaceutically acceptable salt or prodrug thereof and at least one agent activating the intrinsic pathway of apoptosis or a pharmaceutically acceptable salt or prodrug thereof; (ii) a combined preparation as specified herein, and/or (iii) a medicament as specified herein, and
b) thereby inhibiting said lymphoma cell.

The method of the present invention, preferably, is an in vitro method. Moreover, it may comprise steps in addition to those explicitly mentioned above. For example, further steps may relate, e.g., to obtaining and/or culturing a lymphoma cell for step a). Moreover, one or more of said steps may be performed by automated equipment. The method may, however, also be an in vivo method, wherein contacting step a) is performed in vivo on a subject known or suspected to suffer from lymphoma. Thus, the method may also be a method of treating lymphoma in a subject.

The term “contacting” is understood by the skilled person. Preferably, the term relates to bringing pharmaceutically active compounds, preparations, or medicaments into physical contact with a lymphoma cell and thereby allowing the compounds and the lymphoma cell to interact.

The term “inhibiting a lymphoma cell”, as used herein, relates to preventing a lymphoma cell from migrating and/or proliferating. Preferably, the term relates to causing said lymphoma cell to undergo apoptosis. Thus, more preferably, the term relates to killing said lymphoma cell. In case the methodisaninvivomethod, inhibiting a lymphoma cell, preferably, is treating lymphoma as specified herein above.

The term “subject”, as used herein, relates to a vertebrate, preferably a livestock or companion animal, more preferably a mammal, including in particular humans, pigs, cattle, goats, sheep, horses. More preferably, the subject is a human. Preferably, the subject is known or is suspected to suffer from lymphoma, more preferably was diagnosed to suffer from lymphoma.

In view of the above, the following embodiments are particularly envisaged:

1. An NF-κB inhibitor or a pharmaceutically acceptable salt or prodrug thereof for use in treating lymphoma, wherein said use further comprises administration of at least one agent activating the intrinsic pathway of apoptosis.
2. The NF-κB inhibitor for use of embodiment 1, wherein said lymphoma is T-cell lymphoma.
3. The NF-κB inhibitor for use of embodiment 1 or 2, wherein said lymphoma is cutaneous T-cell lymphoma.
4. The NF-κB inhibitor for use of any one of embodiments 1 to 3, wherein said NF-κB inhibitor is dimethyl-fumarate (DMF, Dimethyl (E)-butenedioate, CAS No. 624-49-7).
5. The NF-κB inhibitor for use of any one of embodiments 1 to 4, wherein said NF-κB inhibitor is DMF and is administered at a daily dose of at most 500 mg, preferably at most 450 mg, more preferably at most 400 mg.
6. The NF-κB inhibitor for use of any one of embodiments 1 to 5, wherein said NF-κB inhibitor is DMF and is administered at a daily dose of from 300 mg to 500 mg, preferably of from 350 mg to 475 mg, more preferably of from 375 to 450 mg for at least three, preferably at least four, more preferably at least six, weeks.
7. The NF-κB inhibitor for use of any one of embodiments 1 to 6, wherein said NF-κB inhibitor is DMF and is administered at a daily dose of at most 7 mg/kg body weight, preferably at most 6 mg/kg body weight, more preferably at most 5.5 mg/kg body weight.
8. The NF-κB inhibitor for use of any one of embodiments 1 to 7, wherein said NF-κB inhibitor is DMF and is administered at a daily dose of from 4 mg/kg body weight to 7 mg/kg body weight, preferably of from 4.5 mg/kg body weight to 6 mg/kg body weight, more preferably of from 5 mg/kg body weight to 5.5 mg/kg body weight for at least three, preferably at least four, more preferably at least six, weeks.
9. The NF-κB inhibitor for use of any one of embodiments 1 to 8, wherein said agent activating the intrinsic pathway of apoptosis is an inhibitor of at least one anti-apoptotic member of the Bcl-2 family of proteins.
10. The NF-κB inhibitor for use of any one of embodiments 1 to 9, wherein the agent activating the intrinsic pathway of apoptosis is a BH3 mimetic small molecule inhibitor.
11. The NF-κB inhibitor for use of any one of embodiments 1 to 10, wherein the agent activating the intrinsic pathway of apoptosis is 4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)-Benzamide (ABT-199), (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide (ABT-263), or 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737).
12. The NF-κB inhibitor for use of any one of embodiments 1 to 11, wherein the agent activating the intrinsic pathway of apoptosis is ABT-199.
13. The NF-κB inhibitor for use of any one of embodiments 1 to 12, wherein said NF-κB inhibitor is DMF, wherein said agent activating the intrinsic pathway of apoptosis is ABT-199, ABT-263, or ABT-373, and wherein the molar ratio of agent activating the intrinsic pathway of apoptosis to DMF is at least 0.1, preferably at least 0.12, more preferably at least 0.14, most preferably at least 0.15, for at least three, preferably at least four, more preferably at least six, weeks.
14. The NF-κB inhibitor for use of any one of embodiments 1 to 13, wherein said NF-κB inhibitor is DMF, wherein said agent activating the intrinsic pathway of apoptosis is ABT-199, ABT-263, or ABT-373, and wherein the molar ratio of agent activating the intrinsic pathway of apoptosis to DMF is of from 0.1 to 0.3, preferably of from 0.12 to 0.2, more preferably is about 0.17, most preferably is 0.17, for at least three, preferably at least four, more preferably at least six, weeks.
15. The NF-κB inhibitor for use of any one of embodiments 1 to 14, wherein said NF-κB inhibitor is DMF, wherein said agent activating the intrinsic pathway of apoptosis is ABT-199, and wherein the mass ratio of ABT-199 to DMF is at least 0.75 for at least three, preferably at least four, more preferably at least six, weeks.
16. The NF-κB inhibitor for use of any one of embodiments 1 to 15, wherein said NF-κB inhibitor is DMF, wherein said agent activating the intrinsic pathway of apoptosis is ABT-199, and wherein the mass ratio of ABT-199 to DMF is of from 0.75 to 2, preferably of from 0.75 to 1.25, more preferably is about 1, most preferably is 1, for at least three, preferably at least four, more preferably at least six, weeks.
17. An agent activating the intrinsic pathway of apoptosis or a pharmaceutically acceptable salt or prodrug thereof for use in treating lymphoma, wherein said use further comprises administration of at least one NF-dB inhibitor.
18. The agent activating the intrinsic pathway of apoptosis of embodiment 17 further having a feature of one of the preceding embodiments.
19. A combined preparation for simultaneous, separate or sequential use comprising
a) at least one NF-κB inhibitor or a pharmaceutically acceptable salt or prodrug thereof; and
b) at least one agent activating the intrinsic pathway of apoptosis or a pharmaceutically acceptable salt or prodrug thereof.
20. The combined preparation of embodiment 19 further having a feature of one of the preceding embodiments.
21. The combined preparation of embodiment 19 or 20 for use in medicine, preferably for use in the treatment of lymphoma.
22. A medicament comprising
a) at least one NF-κB inhibitor or a pharmaceutically acceptable salt or prodrug thereof; and
b) at least one agent activating the intrinsic pathway of apoptosis or a pharmaceutically acceptable salt or prodrug thereof and at least one pharmaceutically acceptable carrier.
23. The medicament of embodiment 22 further having a feature of one of the preceding embodiments.
24 The medicament of embodiment 22 or 23 for use in medicine, preferably for use in the treatment of lymphoma.
25. A kit comprising (i) at least one NF-κB inhibitor or a pharmaceutically acceptable salt or prodrug thereof and at least one agent activating the intrinsic pathway of apoptosis or a pharmaceutically acceptable salt or prodrug thereof; (ii) a combined preparation according to any one of embodiments 19 to 21, and/or (iii) a medicament according to any one of embodiments 22 to 24, comprised in a housing.
26. The kit of embodiment 25 further having a feature of one of the preceding embodiments.
27. A method of inhibiting a lymphoma cell, comprising
a) contacting said lymphoma cell with (i) at least one NF-κB inhibitor or a pharmaceutically acceptable salt or prodrug thereof and at least one agent activating the intrinsic pathway of apoptosis or a pharmaceutically acceptable salt or prodrug thereof; (ii) a combined preparation according to any one of embodiments 19 to 21, and/or (iii) a medicament according to any one of embodiments 22 to 24, and
b) thereby inhibiting said lymphoma cell.
28. The method of embodiment 27, wherein said method is a method of treating lymphoma.
29. The method of embodiment 27 or 28 further having a feature of one of the preceding embodiments.
30. A method for identifying a subject suffering from a lymphoma benefiting from a combined treatment with an NF-κB inhibitor or a pharmaceutically acceptable salt or prodrug thereof and an agent activating the intrinsic pathway of apoptosis, comprising determining bcl-2 expression in lymphoma cells of said subject, and identifying a subject as benefiting from the combined treatment if said lymphoma cells are found to express bcl-2.
31. The NF-κB inhibitor for use of any one of embodiments 1 to 16 and/or the agent activating the intrinsic pathway of apoptosis for use of embodiment 17 or 18, wherein said subject was identified to benefit from combined treatment with an NF-κB inhibitor or a pharmaceutically acceptable salt or prodrug thereof and an agent activating the intrinsic pathway of apoptosis according to the method according to embodiment 31.

All references cited in this specification are herewith incorporated by reference with respect to their entire disclosure content and the disclosure content specifically mentioned in this specification.

### FIGURE LEGENDS

FIG. 1: Bcl-2 inhibitors induce cell death in primary patient CTCL cells and CTCL cell lines. (A, B) Specific cell death in primary CD4⁺ cells isolated from 6 healthy donors (control) and 6 patients with Sezary syndrome (patient) upon treatment with different concentrations of ABT-199 (A) or ABT-263 (B) for 24 h. * represents p<0.05 from respective healthy controls. (C, D) Specific cell death in J16, HH and SeAx cells upon treatment with different concentrations of ABT-199 (C) or ABT-263 (D) (n=4, each) for 24 h. * represents p<0.05 from untreated control. (E) Relative Bcl-2 expression measured by qRTPCR in J16, SeAx and HH cells without stimulation. (F) Western Blot analysis of Bcl-2 protein expression in J16, SeAx and HH cells without stimulation.

FIG. 2: Bcl-2 inhibitors and DMF synergistically induce cell death in primary patient CTCL cells and CTCL cell lines. (A) Specific cell death in primary CD4⁺ cells isolated from 5 patients with Sezary syndrome (left), SeAx (middle) or HH cells (right) (n=4 for HH and SeAx) upon treatment with 100 nM ABT-199, 30 μM DMF or the combination of both drugs. * represents p<0.05 from ABT-199 monotreatment. (B) Western Blot analysis of caspase 3 cleavage in SeAx and HH cells (as indicated) after treatment with 100 or 1000 nM ABT-199, 50 μM DMF or the combination of 1000 nM ABT-199 and 50 μM DMF, as indicated. (C) Representative Western Blot analysis of SeAx Bcl-2 expression following AMAXA transfection with different siRNAs against bcl-2 after 24 h. (D) Quantification of signal intensities from Western blots for Bcl-2 in SeAx cells following AMAXA transfection with different siRNAs against Bcl-2 after 24 h (n=4). * represents p<0.05 from scrambled siRNA control. (E) Specific cell death in SeAx cells transfected with different siRNAs against bcl-2 for 24 h and treated with 30 μM DMF for another 24 h (n=4). * represents p<0.05 from scrambled siRNA control.

FIG. 3: Bcl-2 inhibition induces Bax/Bak oligomerization in CTCL cell lines depending on their Bcl-2 activity. (A) Complex formation of Bax and Bak was determined by PLA in SeAx cells either treated with ABT-199, DMF or the combination of both drugs, as indicated. Vehicle treated cells served as control. Bax/Bak association is depicted by green fluorescence; blue fluorescence indicates nuclear staining with Hoechst dye; shown are representative immunofluorescence analyses from z-stacks. (B) Quantification of mean fluorescence intensities of PLA signals (n=4). * represents p<0.05 from untreated control.

FIG. 4: Combined Bcl-2 and NFκB inhibition inhibits CTCL tumor growth and increases survival in an orthotopic SeAx xenograft model. NSG mice were xenografted with SeAx cells i.d. Treatment of transplanted mice was with either vehicle, one treatment of ABT-199, once daily treatment with DMF for 28 days or the combination of a single application of ABT-199 and the 28 days treatment with a daily application of DMF (n=8, each). (A) Median tumor volume upon treatment with vehicle, ABT-199, DMF and the combination. * represents p<0.05 of the combination-treatment versus vehicle-treated control. (B) Percent reduction in tumor growth at day 18 of mice treated with PBS, ABT-199, DMF or combination. * represents p<0.05 from vehicle-treated control. (C) Survival curves of xenografted mice in the different treatment groups, as indicated. Decrease in survival is reflecting the reach of the endpoints defined under Materials and Methods. (D) Percent survival rate of orthotopically xenografted mice treated with vehicle or DMF at day 30, one day after the end of the treatment phase. (E) Mean survival time of the SeAx tumor bearers in the different treatment groups, as indicated. * represents p<0.05 versus vehicle-treated control.

FIG. 5: Combined Bcl-2 and NFκB inhibition HE-morphologically blocks proliferation and induces cell death specifically within CTCL tumors in vivo. SeAx xenograft tumors growing i.d in NSG mice that were treated as described under FIG. 4 were used (A) Representative H&E-stained specimens of primary SeAx tumors from the vehicle control and the combination group (upper panels 20×, lower panels 200×). Red arrows mark necrotic areas. (B) Semiquantitative score of necrotic areas in the primary SeAx xenograft tumors (0-25% tumor area covered by necrosis=0; 25-50% tumor area covered by necrosis=1; 50-75% tumor area covered by necrosis=2; 75-100% tumor area covered by necrosis=3). (C) Quantification of the mitosis count per view under 200× magnification in the four treatment groups.

FIG. 6: Combined Bcl-2 and NFκB inhibition blocks proliferation and induces apoptosis specifically within CTCL tumors in vivo in immunohistochemistry. (A) Representative specimens of primary SeAx tumors from the vehicle control and the combination group. Immunohistochemical detection of cellular positivity for the proliferation marker Ki-67 (400×). (B) Quantification of the Ki-67 positive cell count per view under 400× magnification in the four treatment groups. (C) Representative specimens of primary SeAx tumors from the vehicle control and the combination group. Immunohistochemical detection of cellular positivity for cleaved caspase-3 as a readout for apoptotic activity (400×). (D) Quantification of the cleaved caspase-3-positive cell count per view under 400× magnification in the four treatment groups.

The following Examples shall merely illustrate the invention. They shall not be construed, whatsoever, to limit the scope of the invention.

### EXAMPLE 1: MATERIALS AND METHODS

### 1.1 Patients

Seven patients with Sezary syndrome (CTCL stage IV) diagnosed according to WHO-EORTC classification of CTCL and criteria of the international society of cutaneous lymphomas were included in the study. As controls, we investigated blood samples of healthy donors (n=7). Informed consent was obtained from all subjects before inclusion. The study was conducted according to ethical guidelines at our institution and the Helsinki Declaration and was approved by the ethics committee II of the University of Heidelberg.

### 1.2 Reagents and T Cells

ABT-199, ABT-263 and the mouse antibody against human Bcl-2 were obtained from Santa Cruz Biotechnology. DMF and monomethyl fumarate (MMF) were purchased from Sigma-Aldrich. Caspase-3 and cleaved-Caspase-3 antibodies came from Cell Signaling Technology, Ki-67 antibodies from Novacastra. Primary CD4+ T cells were isolated from heparinized whole blood and treated as indicated (Nicolay et al. (2016), Blood 128(6):805-815). Cell lines were obtained from ATCC (HH, J16) or generously donated by Drs. Gniadecki and Pless from Bispebjerg Hospital Copenhagen, Denmark and Dr. Kaltoft, Aarhus University Denmark (SeAx) or Prof. Eichmuiller from DKFZ Heidelberg, Germany (MyLa), and cultured as described (Nikolay et al, ibd.)

1.3 Cell Death Assays, Quantitative Reverse Transcript (qRT) PCR, Western Blot were Performed as According to Standard Methods.

### 1.4 Proximity Ligation Assay (PLA)

In order to determine protein interactions a proximity ligation assay was performed with Duolink® PLA by Sigma-Aldrich as described before (Schroeder et al. (2017), Nat. Publ. Gr. October 2016:1-12). The rabbit antibody against Bak and the mouse antibody against Bax were obtained from Abcam.

1.5 siRNA Mediated Knock-Down

For the bcl-2 knockdown, the AMAXA® Cell Line Nucleofactor® Kit T (Lonza) was used, and the transfection was performed as described earlier. FlexiTube GeneSolution GS596 siRNAs for Bcl-2 were obtained from Qiagen.

### 1.6 In Vivo Xenograft CTCL Mouse Model and Treatment of Mice

All animal experiments were in accordance with the approved guidelines of the local Governmental Committee for Animal Experimentation (RP Karlsruhe, Germany, license G282/15). Mice were maintained at a 12 h light-dark cycle with unrestricted diet and water. Under isoflurane inhalation anesthesia (1-1.5% in O₂, 0.5 L/min), 3×10⁶ SeAx cells suspended in 30 μl of PBS/Matrigel (1:1, v/v) were injected intradermally (i.d.) into the right flank of 6-7-week-old female NOD SCID gamma (NSG) mice recruited from the Center for Preclinical Research, DKFZ, Heidelberg and kept on Kliba diet 3307 with n=8 mice/group. When tumors reached palpable size (mean of 0.025 cm³) mice were randomized and treated first as indicated once by oral gavage with ABT-199 dissolved in a vehicle of 50% propylenglycol, 30% polyethylenglycol 400 and 10% ethanol at a concentration of 100 mg/kg body weight, 10 μl/g mouse or vehicle alone as indicated. One day later, intraperitoneal (i.p.) treatment with DMF once daily for 28 days was started as indicated. The compound, dissolved at 3 mg/ml in 30° C. pre-warmed PBS, was given with 10 μl/g mouse at a dose of 30 mg/kg bodyweight. For control, PBS (10 μl/g mouse) was injected. Tumor volume was measured with a caliper 2-3 times a week and calculated according to the formula: V=(length (mm)×width (mm)²)/2. Necropsies were taken when one tumor diameter reached 1.5 cm or when mice reached stop criterion of the German Society of Laboratory Animal Sciences (GV-SOLAS), here defined as survival. For tissue staining, the liver and a quarter of the tumor was fixed in 4% PBS-buffered formaldehyde and embedded into paraffin according to routine procedures.

### 1.7 Histologic and Immunohistochemical Stainings

Hematoxylin and eosin staining as well as immunohistochemistry were performed on 5 μm paraffin sections as described. For immunohistochemical stainings, paraffin-embedded tissue was sectioned into 5 μm slices. Ki-67 was detected in deparaffinized tissue specimens after antigen retrieval (20 min at boiling temperature in 10 mM sodium citrate pH 6.0), block of endogenous peroxidases in 3% H₂O₂ in PBS for 10 min, block in 100% goat serum for 1 h at room temperature (RT) and incubation overnight at 4° C. in a 1:250 dilution of anti-Ki-67 antibodies (NCL-Ki67p, Novacastra). Slides were incubated with appropriate horse-radish peroxidase (HRP)-coupled secondary antibody (1:200) for 1 h at RT, incubated with HRP substrate solution (DAB/H₂O₂, Sigma, Munich/Germany). Nuclei were counterstained with hematoxylin for 1 min. Mounting was in Eukitt (R. Langenbrinck, Teningen/Germany). Sections incubated without primary antibody were included as negative controls.

Immunohistochemical assessment of the tissue slides was performed by a dermatologist (JPN) and a pathologist (TA).

### 1.8 Microscopy

For microscopy, the following Nikon objective lenses were used (Nikon, Tokio, Japan): Nikon Plan Apoλ 2×/0.1, Nikon Plan Apoλ 20×/0.75. Microscopy was performed at 22° C. throughout with cryopreserved or paraffin sections. As fluorochromes phycoerythrin (PE), fluorescein-isothiocyanate (FITC) and DAPI were used for red, green and blue stainings, respectively. The micrographs were taken with a Nikon DS-Fi2 camera using the Nikon DS-L3 software.

### 1.9 Statistical Analyses

Data are presented as the mean±s.e.m. Two-sided tests were used throughout and the differences were considered statistically significant at P<0.05. Pairwise (univariate) comparisons were performed using Student's t test or the Mann-Whitney U test as appropriate. Normalizations were performed as described in the Figure legends.

### EXAMPLE 2: RESULTS

### 2.1 Bcl-2 Inhibitors Induce Cell Death in Primary Patient CTCL Cells and CTCL Cell Lines.

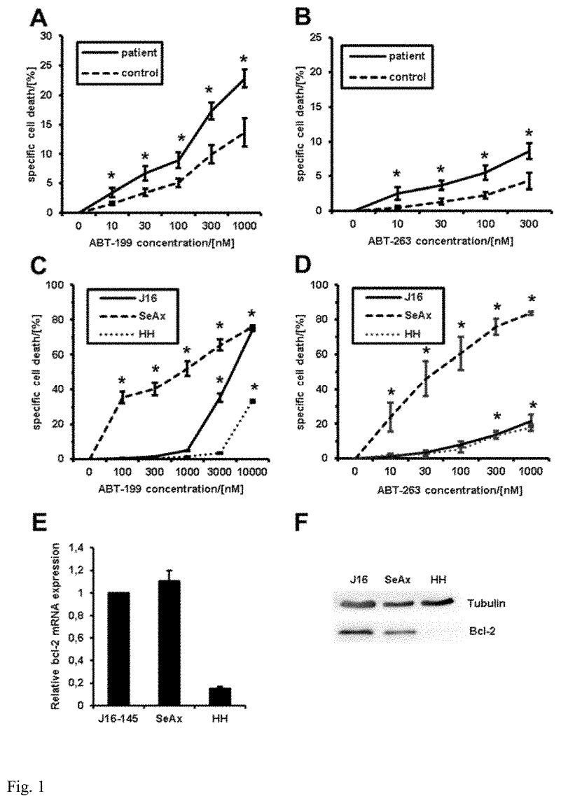

We isolated primary CD4+ CTCL cells from the peripheral blood of seven stage IV CTCL patients and the CD4+ cells from seven healthy donors and treated them with increasing concentrations of the Bcl-2 inhibitors ABT-199 and ABT-263. Both inhibitors induced a dose-dependent specific CD4+ T cell death after 24 h and 48 h with significantly higher rates in Sezary patients' cells than in those from healthy donors (FIGS. 1A-B). This CTCL-specific effect was independent from the tumor burden in blood or skin of the patients or therapeutic regimens applied. To substantiate these results, we also analyzed cell death induction upon Bcl-2 inhibition in established CTCL cell lines SeAx and MyLa. Both showed high cell death sensitivity towards Bcl-2 inhibition, whereas the non-CTCL cell line J16 showed a by far attenuated cell death sensitivity, further corroborating the CTCL-specific effect of Bcl-2 inhibition (FIGS. 1C-D, supplemental FIG. 1). Intriguingly, the CTCL cell line HH showed barely any response to the treatment. In a next step, we set out to correlate the observed cell death rates with the Bcl-2 expression of the cells. Therefore, we quantified Bcl-2 in the tested cell lines on the mRNA level by qRTPCR as well as on the protein level by western blot analysis. We detected almost no expression of Bcl-2 in the HH cell line in comparison to the high Bcl-2 expression in SeAx cells (FIG. 1E,F). Therefore, the observed CTCL cell death induction by Bcl-2 inhibitors correlates with Bcl-2 expression of the respective cells. The weak effect in J16 cells and primary healthy CD4+ T cells confirmed that this cell death effect is specific for malignant CTCL cells and suggested low side effects on benign bystander T cells (FIG. 1A-F).

### 2.2 Bcl-2 Inhibitors and DMF Synergistically Induce Cell Death in Primary Patient CTCL Cells and CTCL Cell Lines.

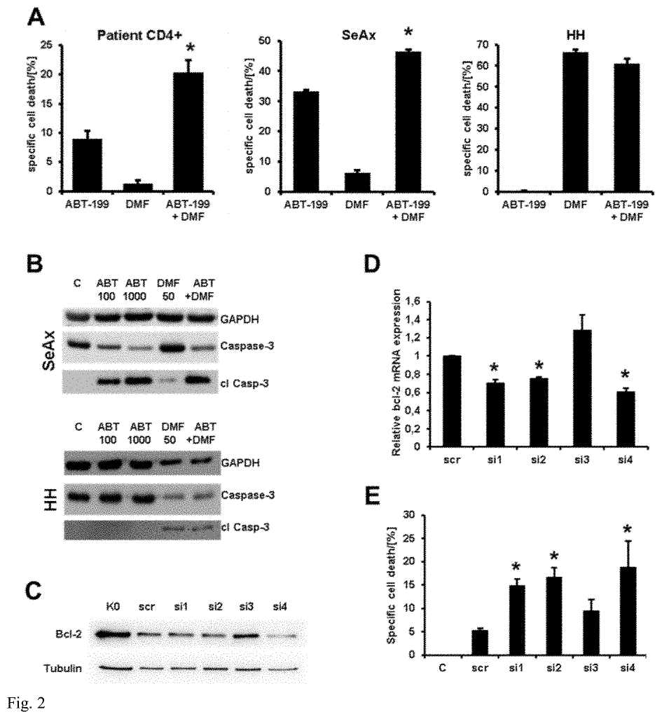

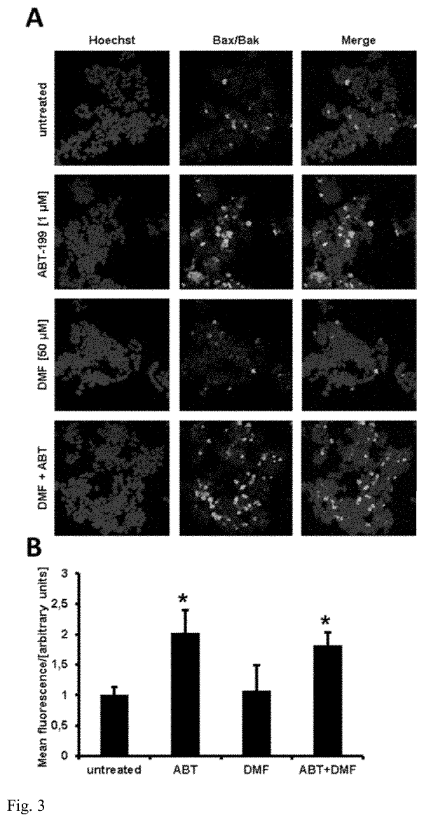

In CTCL cells, the thiol-modifying agent and NFκB inhibitor DMF has been shown to induce apoptosis in CTCL cells in a specific manner. Therefore, we examined a possible synergistic effect of simultaneous DMF-induced NFκB inhibition and Bcl-2 inhibition. Indeed, upon combination treatment, we observed a highly significant cooperative effect of the drugs in isolated primary T cells of Sézary patients that by far exceeded the additive values of both monotreatments (FIG. 2A left panel). Here, both Bcl-2 inhibitors showed similar results. In the CTCL cell line SeAx, we observed a weaker, but still significant over-additive effect with ABT-199 and DMF (FIG. 2A middle panel, supplemental FIG. 3B). Combination treatment of HH cells lacking Bcl-2 expression did not have a similar effect, i.e. the DMF-induced cell death could not be increased by combination with Bcl-2 inhibitors (FIG. 2A right panel, supplemental FIG. 3C).

In order to confirm the apoptotic feature of the observed cell death by combined NFκB and Bcl-2 inhibition, we tested caspase cleavage. We found a dose-dependent caspase 3 cleavage in SeAx cells upon treatment with ABT-199 alone or in combination with DMF. This correlated with cell death measured by flow-cytometry. In contrast, in HH cells only DMF could induce significant caspase 3 cleavage. ABT-199 had no effect, most likely due to the lack of Bcl-2 and the missing cell death induction by ABT-199 (FIG. 2B).

To confirm the synergistic effect of decreasing Bcl-2 and NFκB activity in CTCL cells, we performed a bcl-2 knockdown in SeAx cells. Therefore, the cells were transfected with anti-bcl-2-siRNAs via AMAXA® nucleofection. As shown in FIGS. 2 C and D, transfection with different siRNAs caused decreased Bcl-2 expression levels in cells treated with siRNAs 1, 2 and 4 and ranged from 60% to 75% compared to the level of the control cells treated with scrambled siRNA. 24 hours after transfection, we treated the transfected cells with 30 μM DMF for another 24 h. Cell death upon DMF treatment in this study is illustrated in FIG. 2E. We found a direct correlation between Bcl-2 expression and cell death sensitivity in the transfected cells, i. e. lower Bcl-2 activity lead to higher cell death. Therefore, we concluded that loss of Bcl-2 sensitizes CTCL cells towards other cell death stimuli like NFκB inhibition. This experiment further supported the observed synergistic effect of the combined small-molecule Bcl-2 and NFκB-inhibitors. Mechanistically, the synergistic cell death induction by combined inhibition of Bcl-2 and NFκB is not caused by mutual alterations of their signaling cascade, but rather by blocking two independent pathways. Upon Bcl-2 knockdown, we found no alterations of the IκB-mRNA expression on qRTPCR level. IκB inhibits NFκB subunits via binding in the cytoplasm, and IκBa expression is used as a readout for activity of the transcription factor NFκB, as it represents a target gene of NFκB. This lead to the suggestion that Bcl-2 does not directly regulate NFκB activity. Vice versa, we treated J16, HH and SeAx cells with DMF and its metabolite MMF and measured the protein levels of Bcl-2 by western blot analysis. Here, no influence on the levels of Bcl-2 or other anti-apoptotic Bcl-2 family members like Mcl-1 could be detected in any of the cell lines upon NFκB inhibition. These results strongly suggest that here, the NFκB- and Bcl-2 signaling pathways do not influence or regulate each other directly in their activities.

2.3 Bcl-2 Inhibition Induces Bax/Bak Oligomerization in CTCL Cell Lines Depending on their Bcl-2 Level.

To further elucidate the mechanisms of cell death induction observed upon ABT-199 and DMF treatment, we used a proximity ligation assay (PLA), that indicates the interaction between Bax and Bak at an early step in the signaling cascade leading to apoptosis. With this method, proximity of the two proteins Bax and Bak can be visualized by green fluorescence. If not inhibited, Bcl-2 binds the two proteins at the mitochondrial membrane and, thus, prevents them from oligomerization and pore formation. Upon Bcl-2 inhibition, Bcl-2 should release Bax and Bak so that they can form pores through which cytochrome c leaves the mitochondria and completes the apoptosome. Therefore, the detection of green fluorescence confirms effective Bcl-2 inhibition and Bax/Bak interaction. Indeed, we found a significant increase in green fluorescence upon ABT-199 treatment in SeAx cells. This effect was not further enhanced by DMF treatment (FIG. 3A, B). Thereby we could confirm the direct effect of Bcl-2 inhibition on apoptosis induction in this cell line. As expected, DMF and ABT-199 had no effect on Bax/Bak interaction in HH cells further confirming our previous findings.

### 2.4 Combination of Bcl-2 and NFκB Inhibition Decreases Tumor Growth and Increases Survival In Vivo

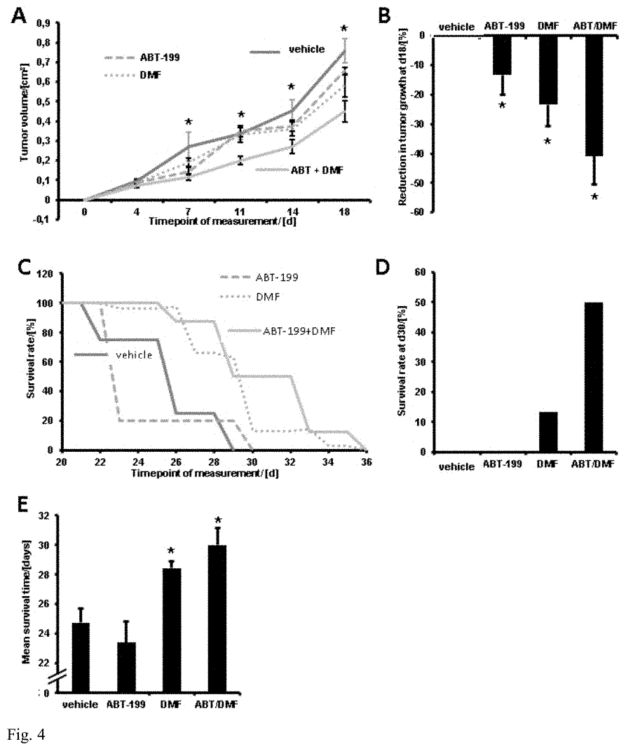

To prove the in vivo relevance of our findings, we used a CTCL xenograft mouse model. We intradermally injected the SeAx cell line into NSG mice. After the detection of intradermal tumor growth, the animals were randomized into 4 therapy groups: one vehicle-treated control group, two monotherapy groups with either ABT-199 or DMF treatment, and a combination therapy group treated with ABT-199 and DMF together (FIG. 4). Within the first 10 days after ABT-199 monotreatment, 3/8 animals dropped out of this group, while in the vehicle group only 1/8 mice dropped out within the first 2 weeks of vehicle application. First, we confirmed the xenograft tumors to consist of SeAx cells by immunohistochemistry. As a primary readout of the therapeutic effect we assessed growth of the xenograft tumors. Upon treatment, we observed a weak, but not significant decrease in tumor growth by both, DMF and ABT-199 monotreatments. The combination of both drugs, however, led to a significant and lasting stable reduction of tumor growth starting already after 7 days of treatment (FIG. 4A). A more than 40% reduction of SeAx xenograft tumors was seen in the DMF/ABT-199 group at day 18, an effect that could never be reached by single treatment (FIG. 4B). In accordance with our in vitro experiments we could confirm an over-additive therapeutic effect of the combination treatment in vivo (FIG. 4A,B). Similar synergistic effects of the combination treatment could be observed in the survival rates of the treated animals. The first animals in the PBS control group had to be euthanasized due to reaching the endpoint already after 22 days, whereas this was necessary in the combination treatment group only after 26 days (FIG. 4C). FIG. 4C shows an overall survival benefit by DMF monotreatment and the combination treatment with DMF and ABT-199. Based on our data, ABT-199 monotreatment shows no advantage with respect to survival (FIG. 4C). At day 30, 50% of the animals in the combination treatment group were still under follow-up compared to 13% in the DMF group and 0% in both other groups. This finding emphasizes the beneficial therapeutic effect of combined DMF and ABT-199 therapy (FIG. 4D). In summary, we could show a significant increase of the mean survival time by more than 20% as result of the combination treatment (FIG. 4E). While Bcl-2 inhibition with ABT-199 did not show sufficient therapeutic effects in the monotreatment in vivo, it was able to strikingly reduce tumor growth and to increase survival rates if used in combination treatment (FIG. 4A-E). In contrast to the HH mouse model we had established before to evaluate DMF monotreatment in vivo⁸, in the present SeAx xenograft model, no spreading of the malignant T cells to distant organs (liver, spleen and lymph nodes) was detectable. Therefore, in this mouse model, only the effects of the treatment on the primary xenograft tumors could be evaluated.

2.5 Combined Bcl-2 and NFκB Inhibition Blocks Proliferation and Induces Cell Death Specifically within CTCL Tumors In Vivo

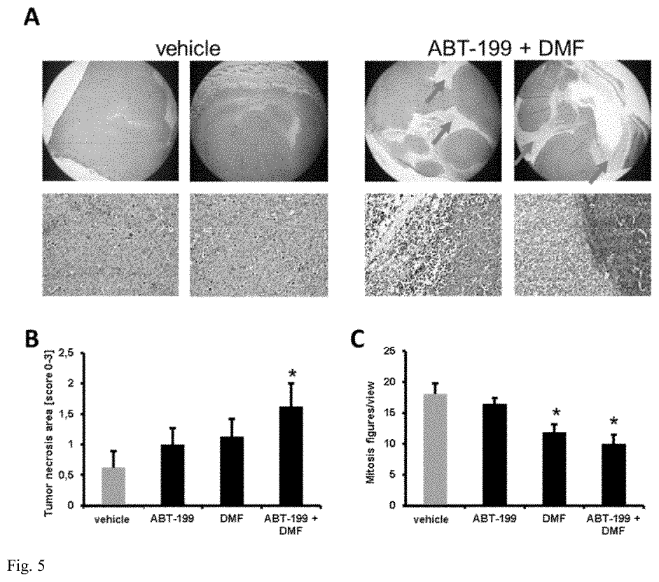

After reaching the end points, the tumors were resected for histological analysis of hematoxylin and eosin (H&E)-stained sections. In contrast to the control group we found significantly larger necrotic tumor areas in the combination group as a sign of tumor cell death and subsequent reduction in tumor size (FIG. 5A). Upon quantification with a semi-quantitative score, we found a tendency towards increased cell death of the tumor cells in the monotreatment group, compared to the vehicle control. This effect reached statistical significance in the combination group. In addition, we counted the mitosis figures in the H&E specimens and we found a significant decrease in mitotic activity upon treatment with DMF. This effect was even more pronounced upon combination treatment. This finding indicates a deceleration of tumor growth by a reduction of CTCL cell proliferation in the xenograft tumors, especially upon combination treatment (FIG. 5C).

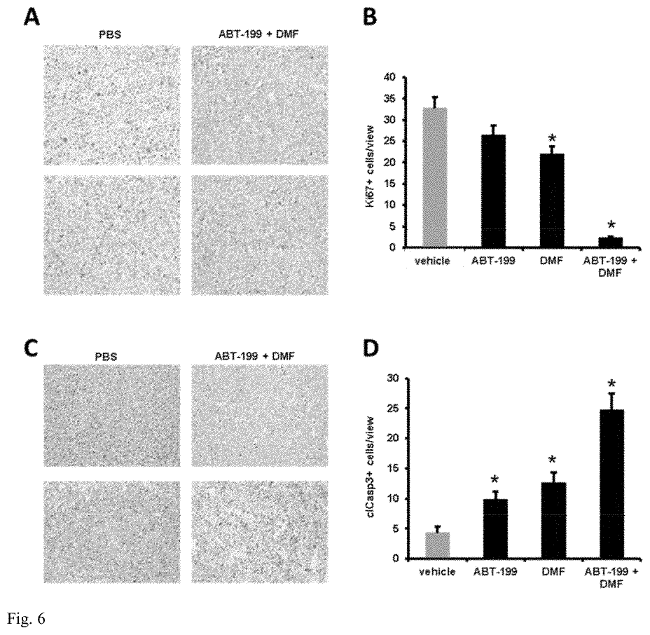

This observation was confirmed by immunohistochemical evaluation of the Ki-67 proliferation index in the tumors. Here, we observed massive proliferative activity in the xenograft tumors of the vehicle controls with up to 33% of the tumor cells positive for Ki-67, especially in the tumor periphery (FIG. 6A, B). The monotreatments with DMF and ABT-199 slightly reduced positivity for Ki-67, whereas in the combination treatment group almost no Ki-67-positive cells were detectable (FIG. 6B). In a further step, we also stained the tumor sections for cleaved caspase 3 as a readout of apoptotic activity (FIG. 6C, D). Indeed, we found slightly increased caspase 3 cleavage within the xenograft tumors upon respective monotreatment with ABT-199 or DMF. However, combination treatment with both drugs resulted in a massive increase in caspase 3 cleavage (FIG. 6C, D). In addition, in the combination-treated tumors we found the cleaved caspase 3+ cells especially close to areas covered by necrosis (FIG. 6C, supplemental FIG. 8).

### LITERATURE

DeYoung at al. (2011), Diabetes Technology & Therapeutics 13:1145
Giam et al., 2009, Oncogene 27 Suppl 1:S128-36
Lessene et al., 2008, Nat Rev Drug Discov 7:989-1000
Nicolay et al. (2016), Blood 128(6):805
Ramazani et al., (2016), Int J Pharm. 499(1-2): 358-367
Schroeder et al. (2017), Nat. Publ. Gr. October 2016:1-12
Tse et al., 2008, Cancer Res 68:3421-8
Walensky et al., 2004, Science 305: 1466
Wendt et al., 2006, J Med Chem 49:1165
Yin et al., 2005, J Am Chem Soc 127(29): 10191

1. A method of inhibiting a lymphoma cell, comprising
a) contacting said lymphoma cell with (i) at least one NF-κB inhibitor or a pharmaceutically acceptable salt or prodrug thereof and at least one agent activating the intrinsic pathway of apoptosis or a pharmaceutically acceptable salt or prodrug thereof, and/or (ii) a combined preparation according to claim 12, and
b) thereby inhibiting said lymphoma cell.

2. The method of claim 1, wherein said lymphoma is cutaneous T-cell lymphoma.

3. The method of claim 1, wherein said NF-κB inhibitor is dimethyl-fumarate (DMF).

4. The method of claim 1, wherein said NF-κB inhibitor is DMF and is administered at a daily dose of at most 500 mg, preferably at most 450 mg, more preferably at most 400 mg.

5. The method of claim 1, wherein said NF-κB inhibitor is DMF and is administered at a daily dose of from 300 mg to 500 mg, preferably of from 350 mg to 475 mg, more preferably of from 375 to 450 mg for at least three, preferably at least four, more preferably at least six, weeks.

6. The method of claim 1, wherein the agent activating the intrinsic pathway of apoptosis is a BH3 mimetic small molecule inhibitor.

7. The method of claim 1, wherein the agent activating the intrinsic pathway of apoptosis is 4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)-Benzamide (ABT-199), (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide (ABT-263), or 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737), preferably is ABT-199.

8. The method of claim 1, wherein said NF-κB inhibitor is DMF, wherein said agent activating the intrinsic pathway of apoptosis is ABT-199, ABT-263, or ABT-373, and wherein the molar ratio of agent activating the intrinsic pathway of apoptosis to DMF is at least 0.1, preferably at least 0.12, more preferably at least 0.14, most preferably at least 0.15, for at least three, preferably at least four, more preferably at least six, weeks.

9. The method of claim 1, wherein said NF-κB inhibitor is DMF, wherein said agent activating the intrinsic pathway of apoptosis is ABT-199, and wherein the mass ratio of ABT-199 to DMF is of from 0.75 to 2, preferably of from 0.75 to 1.25, more preferably is about 1, most preferably is 1, for at least three, preferably at least four, more preferably at least six, weeks.

10. A method for treating lymphoma in a patient in need thereof, the method comprising administering to the patient an NF-κB inhibitor or a pharmaceutically acceptable salt or prodrug thereof, wherein said method further comprises administration of at least one agent activating the intrinsic pathway of apoptosis.

11. A method for treating lymphoma in a patient in need thereof, the method comprising administering to the patient an agent activating the intrinsic pathway of apoptosis or a pharmaceutically acceptable salt or prodrug thereof, wherein said method further comprises administration of at least one NF-κB inhibitor.

12. A combined preparation for simultaneous, separate or sequential use comprising
a) at least one NF-κB inhibitor or a pharmaceutically acceptable salt or prodrug thereof; and
b) at least one agent activating the intrinsic pathway of apoptosis or a pharmaceutically acceptable salt or prodrug thereof.

13. A medicament comprising
a) at least one NF-κB inhibitor or a pharmaceutically acceptable salt or prodrug thereof; and
b) at least one agent activating the intrinsic pathway of apoptosis or a pharmaceutically acceptable salt or prodrug thereof
and at least one pharmaceutically acceptable carrier.

14. A method for treating lymphoma in a patient in need thereof, the method comprising administering to the patient the combined preparation of claim 12.

15. A kit comprising (i) at least one NF-κB inhibitor or a pharmaceutically acceptable salt or prodrug thereof and at least one agent activating the intrinsic pathway of apoptosis or a pharmaceutically acceptable salt or prodrug thereof, and/or (ii) a combined preparation according to claim 12, comprised in a housing.
